(19) United States
(12) Patent Application Publication
(10) Pub. No.: US 2006/0183123 A1
(43) Pub. Date: Aug. 17, 2006

(54) Polymerase-based protocols for the introduction of combinatorial deletions...

(76) Inventor: John C. Salerno, Averill Park, NY (US)

Correspondence Address: ELMORE PATENT LAW GROUP, PC, 209 MAIN STREET, N. CHELMSFORD, MA, 01863, US

(21) Appl. No.: 10/544,419

(22) PCT Filed: Feb. 6, 2004
(86) PCT No.: PCT/US04/03501

§ 371 Date: Jan. 12, 2006

Related U.S. Application Data
(60) Provisional application No. 60/445,689, filed on Feb. 6, 2003.
(60) Provisional application No. 60/445,703, filed on Feb. 6, 2003.
(60) Provisional application No. 60/446,045, filed on Feb. 6, 2003.
(60) Provisional application No. 60/445,704, filed on Feb. 6, 2003.
(60) Provisional application No. 60/474,063, filed on May 29, 2003.

(51) Int. Cl. C40B 40/08; C12P 19/34

(52) U.S. Cl. 435/6; 435/91.2

(57) ABSTRACT

The present invention relates to improved methods of introducing combinatorial mutations in a region of DNA without the need for subcloning. The invention comprises polymerase-based, mutagenesis methods which may be adapted for use with commercially available mutagenesis kits to generate combinatorial mutations of a region of DNA in a quick, efficient and cost-effective manner. Major applications of this method include vaccine production, directed evolution and other areas that benefit from the development of diversity.

### RELATED APPLICATIONS

This application claims the benefit of U.S. Provisional Application No. 60/445,704, filed on Feb. 6, 2003, U.S. Provisional Application No. 60/445,689, filed on Feb. 6, 2003, U.S. Provisional Application No. 60/445,703, filed on Feb. 6, 2003, U.S. Provisional Application No. 60/446,045, filed on Feb. 6, 2003, and U.S. Provisional Application No. 60/474,063, filed on May 29, 2003, Docket No. RPI-812, entitled “Parental Suppression via Polymerase-based Protocols for the Introduction of Deletions and Insertions.” The entire teachings of the above applications are incorporated herein by reference.

### BACKGROUND OF THE INVENTION

In recent years a number of methods have come into common use that allow the generation of site directed mutants without subcloning based on polymerase activity. This technology is mature enough to allow the sale of a number of mutagenesis kits that are capable of producing point mutants and in some case insertion and deletion mutants (‘indels’).

One such mutagenesis system is supplied by Stratagene (La Jolla, Calif.) and is sold under the name QuikChange® Site Directed Mutagenesis Kit (QCM). The Stratagene system is widely used and effective for the production of single codon mutations. Other examples include Altered Sites® II in vitro Mutagenesis System (Promega Corporation, Madison, Wis.) and BD Transformer™ Site-Directed Mutagenesis Kit (BD BioSciences, Palo Alto, Calif.).

None of the currently available commercial kits or other known site-directed mutagenesis procedures provide for the production of site-directed combinatorial mutations to provide combinatorial numbers of site-directed mutations in a quick, efficient and cost effective manner. Combinatorial mutations have important applications in research, medicine, and biotechnology whenever the generation of local diversity is of special importance. A major application is in the production of vaccines which convey immunity to multiple forms of a pathogen. An important obstacle to effective immunization against viruses, for example, is the ability of some viruses to mutate rapidly at a limited number of epitopes which are the exposed binding sites for antibodies. A potential avenue of attack would produce an ensemble of antigens which anticipate all likely mutants in key epitopes. Combinatorial mutation strategies can readily produce an ensemble of recombinant viral proteins at any desired level of local diversity. This ensemble can in principle immunize against existing viral strains and at the same time provide immunity against anticipated mutants which do not yet exist.

### SUMMARY OF THE INVENTION

The present invention relates to improved methods of introducing combinatorial mutations in a region of DNA without the need for subcloning. The invention comprises polymerase-based, mutagenesis methods which may be adapted for use with commercially available mutagenesis kits to generate combinatorial mutations of a region of DNA in a quick, efficient and cost-effective manner. The invention also provides for kits for combinatorial site-directed mutagenesis. The kits of the invention contain reagents and instructions required for carrying out the methods of the invention. Major applications of this method include vaccine development, directed evolution and other areas that benefit from the development of diversity.

### BRIEF DESCRIPTION OF THE DRAWINGS

The foregoing and other objects, features and advantages of the invention will be apparent from the following more particular description of the preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a diagram of a first aspect of the invention.

FIG. 2 is a diagram of a second aspect of the invention.

### DETAILED DESCRIPTION OF THE INVENTION

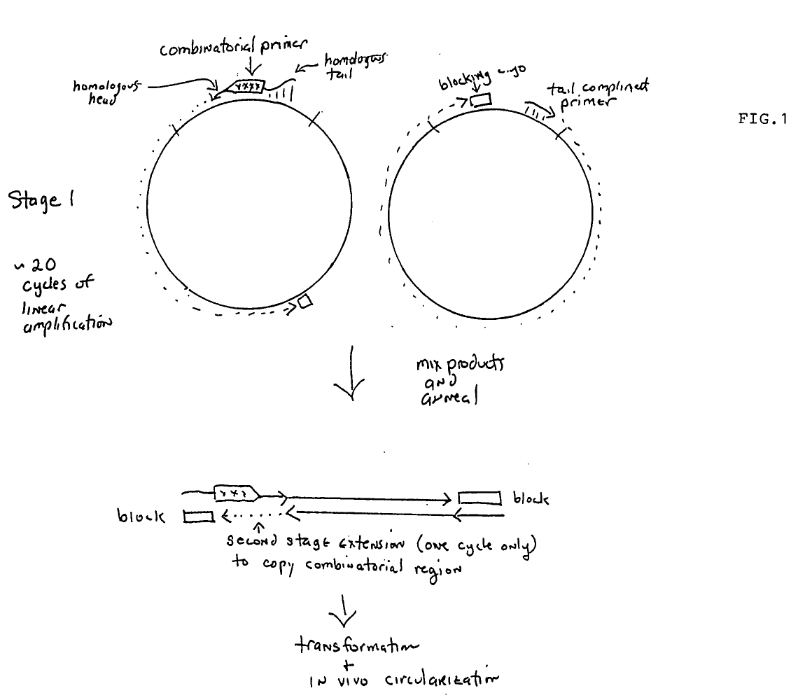

The present invention provides novel, improved methods for the introduction of combinatorial mutations in a region of a DNA sequence of interest without the need for subcloning. In a first aspect of the invention as shown in FIG. 1, the method of introducing combinatorial mutations into a target DNA sequence of interest comprises the steps of:

(a) adding in a first reaction, a combinatorial mutagenic primer and a first blocking oligonucleotide to a first parental DNA having a mutation target site, wherein the combinatorial mutagenic primer comprises a variable mutagenic region flanked by a 3′ region that is complementary to a portion of the first parental DNA and a 5′ invariant tail region that is complementary to a portion of the first parental DNA and wherein the first blocking oligonucleotide halts primer extension prior to the 5′ invariant tail region;

(b) adding in a separate, second reaction, a complementary primer and a second blocking oligonucleotide to a second parental DNA that is identical to the first parental DNA, wherein the complementary primer is complementary to the 5′ invariant tail region of the combinatorial mutagenic primer and wherein the second blocking oligonucleotide halts primer extension prior to the mutation target site of the second parental DNA;

(c) synthesizing in the first reaction, by means of at least one cycle of a single-primer linear amplification reaction, a first DNA strand comprising the combinatorial mutagenesis primer having a variable mutagenic region;

(d) synthesizing in the second reaction, by means of at least one cycle of a single-primer linear amplification reaction, a second DNA strand comprising the complementary primer but not comprising a region that is complementary to the variable mutagenic region of the first DNA strand;

(e) combining the reaction products from (c) with the reaction products from (d);

(f) annealing the first DNA strand to the second DNA strand to form a partially double-stranded DNA intermediate; and

(g) extending by means of at least one cycle of a primer extension reaction, the second DNA strand of the partially double-stranded DNA intermediate to copy the variable mutagenic region of the first DNA strand in the presence of third and fourth blocking oligonucleotides, thereby forming a combinatorial DNA duplex comprising a variable mutagenic region and further comprising overhanging sticky ends. The resulting combinatorial DNA duplex can be used to transform competent or preferably, ultracompetent cells. In one embodiment, the invention optionally comprises the use of selection enzymes to digest the first and second parental DNA after the respective synthesis steps. In another embodiment, the method optionally comprises combining a ligase with the combinatorial DNA duplex of step (g) to facilitate nick repair and recircularization prior to transformation. This requires that the blocking oligonucleotide be constructed to prevent ligation at its 5′ end as well as extension at its 3′ end.

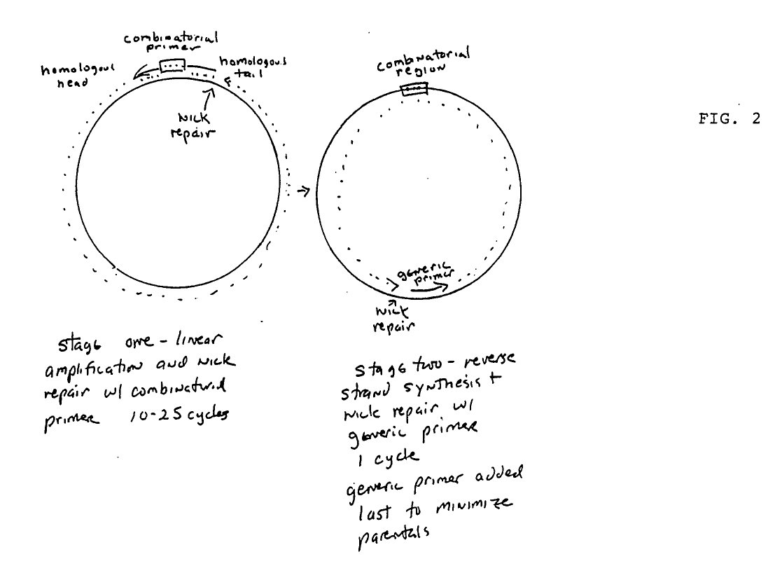

In a second aspect of the invention as shown in FIG. 2, the method of generating combinatorial mutations of a DNA sequence of interest comprises the steps of:

(a) adding in a first reaction, a combinatorial mutagenic primer to a parental DNA having a mutation target site, wherein the combinatorial mutagenic primer comprises a variable mutagenic region that corresponds to the mutation target site and wherein the variable mutagenic region is flanked by a 3′ region that is complementary to a portion of the first parental DNA and a 5′ invariant tail region that is complementary to a portion of the first parental DNA;

(b) synthesizing in the first reaction, by means of at least one cycle of a single-primer linear amplification reaction, a DNA strand that is fully complementary to the parental DNA and comprises the variable mutagenic region;

(c) reacting a ligase with the DNA strand to repair nicks and to recircularize the DNA strand after each cycle of the single-primer linear amplification reaction carried out in step (b);

(d) adding a reverse generic primer to the DNA strand of step (c);

(e) synthesizing by means of at least one cycle of primer extension, the reverse complementary strand of the DNA strand; and

(f) ligating the reverse complementary strand thereby forming a combinatorial DNA duplex comprising a variable mutagenic region.

In one embodiment, an optional digestion step is carried out after the synthesizing step to digest the parental DNA.

The terms “linear amplification reaction,” and “single-primer linear amplification reaction” as used herein, refer to a variety of enzyme mediated polynucleotide synthesis reactions that employ primers to linearly amplify a given polynucleotide and proceeds through one or more cycles, each cycle resulting in polynucleotide replication. A linear amplification reaction cycle typically comprises the steps of denaturing the double-stranded parental template, annealing the single primer or primers to the denatured template and synthesizing oligonucleotides from the primers. Thus the term “linear amplification reaction” as used herein is meant to include all of these steps. In the case of a single-primer linear amplification reaction, only one primer is used in each separate single-primer linear amplification reaction. Linear amplification reactions used in the methods of the invention differ significantly from the polymerase chain reaction (PCR). The polymerase chain reaction produces an amplification product that grows exponentially in amount with respect to the number of cycles. Linear cyclic amplification reactions differ from PCR because the amount of amplification product produced in a linear cyclic amplification reaction is linear with respect to the number of cycles performed. This difference in reaction product accumulation rate laws results from the products of linear amplification schemes not being templates for synthesis of new strands. As in PCR, a linear amplification reaction cycle typically comprises the steps of denaturing the double-stranded template, annealing the single primer or primers to the denatured template, and synthesizing polynucleotides from the primers. The cycle may be repeated several times so as to produce the desired amount of newly synthesized polynucleotide product. Although linear amplification reactions differ significantly from PCR, guidance in performing the various steps of linear cyclic amplification reactions can be obtained from reviewing literature describing PCR and other polymerase based methods including, PCR: A Practical Approach, M. J. McPherson, et al., IRL Press (1991), PCR Protocols: A Guide to Methods and Applications, by Innis, et al., Academic Press (1990), and PCR Technology: Principals and Applications of DNA Amplification, H. A. Erlich, Stockton Press (1989). PCR is also described in many U.S. Patents, including U.S. Pat. Nos. 4,683,195, 4,683,202; 4,800,159; 4,965,188; 4,889,818; 5,075,216; 5,079,352; 5,104,792, 5,023,171; 5,091,310; and 5,066,584, which are hereby incorporated by references. Many variations of amplification techniques are known to the person of skill in the art of molecular biology. These variations include rapid amplification of DNA ends (RACE-PCR), amplification refectory mutation system (ARMS), PLCR (a combination of polymerase chain reaction and ligase chain reaction), ligase chain reaction (LCR), self-sustained sequence replication (SSR), Q-beta amplification, and stand displacement amplification (SDA), and the like. A person of ordinary skill in the art may use these methods to modify the linear amplification reactions used in the methods of the invention.

The terms “parental DNA”, “first parental DNA” and “second parental DNA” as used herein refer to DNA vectors comprising a region that is targeted for combinatorial mutation referred to herein as the “mutation target site”. The parental DNAs serve as templates for the linear amplification reactions in accordance with the methods of the invention.

A ‘combinatorial mutation primer’ actually comprises a set of primers which have identical or nearly identical flanking regions and a central “variable mutagenic region”; for example, a combinatorial set might have all possible amino acid combinations of three central codons, comprising 8,000 different primers each with unique variable regions but each having identical flanking regions. Each of the primers in the combinatorial set has the potential to create a corresponding number of different mutagenized DNA thereby resulting in combinatorial numbers of mutagenized DNA. As used herein, the term “combinatorial” refers to at least about, 10, preferably at least about 20, preferably at least about 50, and more preferably, at least about 100. The variable mutagenic region of the combinatorial mutagenic primer is flanked at the 3′ end by a head region that is complementary to a portion of the parental DNA, and an invariant 5′ tail region that is complementary to a portion of the parental DNA. The combinatorial mutagenic primer is designed such that the head region and the tail region are complementary to those portions of the parental DNA that flank the region where site-directed mutagenesis is desired (referred to herein as the “mutation target site”) of the parental DNA.

Combinatorial numbers of mutants and ‘limited chimera’ can be constructed with a limited number of primers by applying the multiple mutation approach with mixtures of mutagenic primers. (The chimera produced are limited in scope by the size of the individual primers used). For example, n sets consisting of m mutagenic primers each, binding to n different sites within a gene, would generate mⁿ mutants from mⁿ primers when run together in the first stage. A single generic primer would suffice for the second stage. Use of a combinatorial mutagenic primer (a primer set in which all or many possible combinations of bases in a short stretch are present) would produce a combinatorial mixture of mutants concentrated in a single site. Since in all cases the mutants are produced without subcloning and transform directly into cell lines capable of expression, the system has great potential for selection-based applications.

The “complementary primer” as used herein refers to a primer that is complementary to a portion of the parental DNA and that is also complementary to the invariant 5′ tail region of the combinatorial mutagenic primer. The combinatorial mutagenic primer and the complementary primer are each designed such that the variable mutagenic region of the combinatorial mutagenic primer and the complementary primer do not overlap, but are contiguous or nearly so.

In accordance with the first aspect of the invention, the combinatorial mutagenic primer and a first blocking oligonucleotide are added to a first parental DNA in a first reaction and the complementary primer and a second blocking oligonucleotide are added to a second parental DNA in a separate, second reaction. At least one cycle, and preferably about 20 to 25 cycles, of single-primer linear amplification is carried out in each of the first and second reactions. As discussed above, the first parental DNA in the first reaction is identical to the second parental DNA in the second reaction. The first blocking oligonucleotide in the first reaction is complementary to any region downstream from the head region of the combinatorial mutagenic primer so long as the blocking oligonucleotide stops extension of the combinatorial mutagenic primer prior to 3′ end of the combinatorial site of the first parental DNA. The second blocking oligonucleotide in the second reaction is complementary to the region of the parental DNA prior to the 3′ end of the combinatorial site of the second parental DNA. The purpose of the second blocking oligonucleotide in the second reaction is to prevent the complementary primer from extending through the combinatorial site of the parental DNA during the single-primer linear amplification reaction. Thus, when the reaction product of the first reaction (referred to herein as the “first DNA strand” comprising the combinatorial mutagenic primer having a variable mutagenic region, but not extending the full length of the first parental DNA) is combined and annealed with the reaction product of the second reaction (referred to herein as the “second DNA strand” comprising the complementary primer but not comprising a region complementary to the variable mutagenic region of the first DNA strand) a partially double-stranded DNA intermediate is formed. This DNA intermediate is only partially double-stranded because the variable mutagenic region of the first DNA strand is not duplexed with a corresponding complementary region of the second DNA strand.

At least one cycle, and preferably only one cycle, of a second stage primer extension reaction is then carried out on the partially double-stranded DNA intermediate to extend the second DNA strand to copy the variable mutagenic region of the first DNA strand thereby forming a combinatorial DNA duplex comprising a variable mutagenic region. Optionally, third and fourth blocking oligonucleotides can be added to the primer extension reaction to preserve overhanging sticky ends on the combinatorial DNA duplex that are suitable for in vivo recircularization and nick repair upon transformation of the combinatorial DNA duplex into a host cell.

In accordance with the second aspect of the invention, the combinatorial mutagenic primer is added to a parental DNA. At least one cycle, and preferably about 20 to 25 cycles, of single-primer linear amplification is carried out to synthesize a DNA strand comprising the combinatorial mutagenic primer and the remaining portion of the parental DNA. After each cycle of linear amplification, the first DNA strand is reacted with a ligase for nick repair and recircularization of the first DNA strand. A reverse generic primer is added to the DNA strand and preferably only one cycle of primer extension is carried out to extend the reverse generic primer to synthesize the reverse complementary strand of the DNA strand. The reverse complementary strand is reacted with a ligase for nick repair thereby forming a combinatorial DNA duplex comprising a variable mutagenic region.

It is understood by one skilled in the art that although reference is made herein to “a combinatorial DNA duplex comprising a variable mutagenic region”, what results from the methods of the invention are in fact combinatorial numbers of mutagenized DNA each with a unique variable mutagenic region corresponding to the combinatorial mutagenic primer set as discussed above.

The host cells used for transformation in accordance with the invention may be prokaryotic or eukaryotic. Preferably the host cells are prokaryotic, more preferably, the host cells for transformation are E. coli cells. Techniques for preparing and transforming competent single cell microorganisms are well know to the person of ordinary skill in the art and can be found, for example, in Sambrook et al., Molecular Cloning: A Laboratory Manual Coldspring Harbor Press, Coldspring Harbor, N.Y. (1989), Harwood Protocols For Gene Analysis, Methods In Molecular Biology Vol. 31, Humana Press, Totowa, N.J. (1994), and the like. Frozen competent cells may be transformed so as to make the methods of the invention particularly convenient.

The term “oligonucleotide” as used herein with respect to the mutagenic primer the complementary primer and the blocking oligonucleotides is used broadly. Oligonucleotides include not only DNA but various analogs thereof. Such analogs may be base analogs and/or backbone analogs, e.g., phosphorothioates, phosphonates, and the like. Techniques for the synthesis of oligonucleotides, e.g., through phosphoramidite chemistry, are well known to the person ordinary skilled in the art and are described, among other places, in Oligonucleotides and Analogues: A Practical Approach, ed. Eckstein, IRL. Press, Oxford (1992). Preferably, the oligonucleotide used in the methods of the invention are DNA molecules.

In accordance with an alternative embodiment of the invention, the parental target DNA used as templates during the first stage linear amplification reactions can optionally be digested to facilitate increased mutation frequency (by suppressing the parental target DNA). The term “digestion” as used herein in reference to the enzymatic activity of a selection enzyme is used broadly to refer both to (i) enzymes that catalyze the conversion of a polynucleotide into polynucleotide precursor molecules and to (ii) enzymes capable of catalyzing the hydrolysis of at least one bond on polynucleotides so as to interfere adversely with the ability of a polynucleotide to replicate (autonomously or otherwise) or to interfere adversely with the ability of a polynucleotide to be transformed into a host cell. Restriction endonucleases are an example of an enzyme that can “digest” a polynucleotide. Typically, a restriction endonuclease that functions as a selection enzyme in a given situation will introduce specific single cleavages into the phosphodiester backbone of the template strands that are digested.

The term “selection enzyme” refers to an enzyme capable of catalyzing the digestion of a parental DNA template for mutagenesis, but not significantly digesting newly synthesized mutagenized DNA strands. Examples of selection enzymes include restriction endonucleases. One suitable selection enzyme for use in the parental strand digestion step is the restriction endonuclease Dpn I, which cleaves the polynucleotide sequence GATC only when the adenine is methylated (6-methyl adenine. Suitable selection enzymes are provided with commercially available mutagenesis kits such as the QuikChange® Site Directed Mutageneisis System kit supplied by Stratagene (La Jolla, Calif.), Altered Sites® II in vitro Mutagenesis System (Promega Corporation, Madison, Wis.) and BD Transformer™ Site-Directed Mutagenesis Kit (BD BioSciences, Palo Alto, Calif.).

In one embodiment of the invention, the invention may be carried out using the reagents provided in commercially available mutagenesis kits such as Stratagene's QuikChange® II XL Site Directed Mutagenesis Kit, Altered Sites® II in vitro Mutagenesis System (Promega Corporation, Madison, Wis.) and BD Transformer™ Site-Directed Mutagenesis Kit (BD BioSciences, Palo Alto, Calif.). However, the present invention may be practiced without the use of a commercially available kit so long as a high fidelity polymerase and high competence cells are used in the present method.

In accordance with the invention, the primers are about 20-50 bases in length, more preferably about 25 to 45 bases in length. However, in certain embodiments of the invention, it may be necessary to use primers that are less than 20 bases or greater than 50 bases in length so as to obtain the mutagenesis result desired. The primers may be of the same or different lengths. In some embodiments, 5′phosphorylation of the primers is be desirable. 5′ phosphorylation may be achieved by a number of methods well known to a person of ordinary skill in the art, e.g., T-4 polynucleotide kinase treatment.

Preferably, the variable mutagenic region of the combinatorial mutagenic primers of the invention are flanked by about 10-15 bases of correct, i.e., non-mismatched, sequence so as to provide for the annealing of the primer to the template DNA strands for mutagenesis. In preferred embodiments of subject methods, the GC content of mutagenic primers is at least 40%, so as to increase the stability of the annealed primers. Preferably, the first and second mutagenic primers are selected so as to terminate in one or more G or C bases. Very high GC content (over 70%), or runs of more than five successive GC bases, are not desirable since this decreases specificity.

The single-primer linear cyclic amplification reaction may be catalyzed by a thermostable or non-thermostable high-fidelity polymerase. Polymerases for use in the linear cyclic amplifcation reactions of the subject methods have the property of not displacing the primers that are annealed to the template. Preferably, the polymerase used is a thermostable polymerase. The polymerase used may be isolated from naturally occurring cells or may be produced by recombinant DNA technology. The use of Pfu DNA polymerase (Stratagene, La Jolla, Calif.), a DNA polymerase naturally produced by the thermophilic archae Pyrococcus furiosus, is particularly preferred for use in the linear cyclic amplification reaction steps of the claimed invention. Other high fidelity polymerases suitable for use in the present invention include but are not limited to, KOD HiFi (EMD Biosciences Inc., Madison, Wis.), Phusion™ High Fidelity Polymerase (Finnzymes, Espoo, Finland).

Single-primer linear amplification reactions as employed in the methods of the invention are preferably carried out to the limits imposed by the polymerase properties and the amplification conditions. Preferably the number of cycles in the linear cyclic amplification reaction step is at least 10 cycles, more preferably at least 20 cycles, and even more preferably at least 25 or more cycles. The limitation on the optimum cycle number is specific to the application and is imposed by runaway PCR artifact triggered by low probability non specific binding. This is a problem primarily in very CG rich regions, and can be overcome by decreasing the number of cycles and increasing the template concentration.

Appropriate reaction conditions are maintained throughout the various stages of the invention to maximize desirable reaction products while minimizing the production of artifact.

The optional digestion step involves the addition of a selection enzyme that is capable of digesting the parental DNA used as templates but does not significantly digest newly synthesized DNA produced during a linear cyclic amplification reaction. By performing the digestion step, the number of transformants containing non-mutagenized DNA is reduced.

In a third aspect of the invention, the methods of the invention are used to produce an ensemble of recombinant viral proteins at any desired level of local diversity. This ensemble can immunize against existing viral strains and at the same time provide immunity against anticipated mutants which do not yet exist. In accordance with one embodiment of this third aspect of the invention, a method for producing combinatorial arrays of viral proteins for use in vaccine production comprises the steps of:

(a) adding in a first reaction, a combinatorial mutagenic primer and a first blocking oligonucleotide to a first parental DNA having a mutation target site that corresponds to a viral protein, wherein the combinatorial mutagenic primer comprises a variable mutagenic region flanked by a 3′ region that is complementary to a portion of the first parental DNA and a 5′ invariant tail region that is complementary to a portion of the first parental DNA and wherein the first blocking oligonucleotide halts primer extension prior to the 5′ invariant tail region;

(b) adding in a separate, second reaction, a complementary primer and a second blocking oligonucleotide to a second parental DNA that is identical to the first parental DNA, wherein the complementary primer is complementary to the 5′ invariant tail region of the combinatorial mutagenic primer and wherein the second blocking oligonucleotide halts primer extension prior to the mutation target site of the second parental DNA;

(c) synthesizing in the first reaction, by means of at least one cycle of a single-primer linear amplification reaction, a first DNA strand comprising the combinatorial mutagenesis primer having a variable mutagenic region;

(d) synthesizing in the second reaction, by means of at least one cycle of a single-primer linear amplification reaction, a second DNA strand comprising the complementary primer but not comprising a region that is complementary to the variable mutagenic region of the first DNA strand;

(e) combining the reaction products from (c) with the reaction products from (d);

(f) annealing the first DNA strand to the second DNA strand to form a partially double-stranded DNA intermediate;

(g) extending by means of at least one cycle of a primer extension reaction, the second DNA strand of the partially double-stranded DNA intermediate to copy the variable mutagenic region of the first DNA strand in the presence of third and fourth blocking oligonucleotides, thereby forming a mutagenized combinatorial DNA duplex comprising a variable mutagenic region and further comprising overhanging sticky ends;

h) transforming a host cell with the mutagenized combinatorial DNA duplex comprising a variable mutagenic region; and

(i) expressing an array of viral proteins encoded by the mutagenized combinatorial DNA duplex.

In accordance with another embodiment of this third aspect of the invention, a method for producing combinatorial arrays of viral proteins for use in vaccine production comprises the steps of:

(a) adding in a first reaction, a combinatorial mutagenic primer to a parental DNA having a mutation target site, wherein the combinatorial mutagenic primer comprises a variable mutagenic region that corresponds to the mutation target site of a viral protein and wherein the variable mutagenic region is flanked by a 3′ region that is complementary to a portion of the first parental DNA and a 5′ invariant tail region that is complementary to a portion of the first parental DNA;

(b) synthesizing in the first reaction, by means of at least one cycle of a single-primer linear amplification reaction, a DNA strand that is fully complementary to the parental DNA and comprises the variable mutagenic region;

(c) reacting a ligase with the DNA strand to repair nicks and to recircularize the DNA strand after each cycle of the single-primer linear amplification reaction carried out in step (b);

(d) adding a reverse generic primer to the DNA strand of step (c);

(e) synthesizing by means of at least one cycle of primer extension, the reverse complementary strand of the DNA strand;

(f) ligating the reverse complementary strand thereby forming a combinatorial DNA duplex comprising a variable mutagenic region;

(h) transforming a host cell with the mutagenized combinatorial DNA duplex comprising a region of variable mutation; and

(i) expressing an array of proteins encoded by the combinatorial DNA duplex.

Another yet another aspect of the invention, a kit is provided for performing the combinatorial mutagenesis methods of the invention. The kits of the invention provide one or more of the enzymes or other reagents for use in performing the subject methods. The kits may contain reagents in pre-measured amounts so as to ensure both precision and accuracy when performing the subject methods. Kits may also contain instructions for performing the methods of the invention. In one embodiment, kits of the invention comprise at least one polymerase and instructions for carrying out the method. The kits may also comprise a DNA vector comprising a cloning site, ultracompetent cells and blocking oligonucleotides complementary to regions of the DNA vector. Kits of the invention may also comprise individual nucleotide triphosphates, mixtures of nucleoside triphosphates (including equimolar mixtures of dATP, dTTP, dCTP and dGTP), and concentrated reaction buffers. In a preferred embodiment, the kits comprise at least one DNA polymerase, concentrated reaction buffer, a nucleoside triphosphate mix of the four primary nucleoside triphosphates in equal amounts, frozen competent or ultracompetent cells and instructions for carrying out the method.

One skilled in the art will appreciate the many advantages that the method of the invention provides. For example, the improved site-directed mutagenesis methods of the invention are useful in protein and enzyme engineering technologies for the production of industrial proteins and enzymes such as detergent enzymes, enzymes useful for neutralizing contaminants, and enzymes suitable for improved or novel biosynthesis of compounds in industry, biotechnology, and medicine. Likewise the methods of the invention are useful in protein engineering technologies for the production of proteins useful in the food and life sciences industries such as primary and secondary metabolites useful in the production of antibiotics, proteins and enzymes for the food industry (bread, beer), and combinatorial arrays of proteins for use in generating multiple epitopes for vaccine production. Combinatorial mutagenesis of key epitopes could anticipate mutations which allow viruses to evade immune response.

The methods of the invention can also be used in the production of mutagenized fusion proteins. A DNA sequence targeted for mutagenesis is tagged or fused with the DNA sequence encoding a known protein (e.g. MBP or GFP). For example, vectors with a GFP gene adjacent to a cloning site would allow easy conversion of a vector for expression of a target gene to one of several possible target gene-GFP mutants with different linkers. These in turn could be targeted to different cell locations by modifications of the opposite (usually N) terminus. Kits designed identify fusion proteins are advantageously used to identify and isolate the mutagenized protein of interest in vitro (western blots) or in tissue using GFP fluorescence.

The patent and scientific literature referred to herein establishes the knowledge that is available to those with skill in the art. All United States patents and published or unpublished United States patent applications cited herein are hereby incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of introducing combinatorial mutations in a DNA sequence of interest, the method comprising the steps of:
(a) adding in a first reaction, a combinatorial mutagenic primer and a first blocking oligonucleotide to a first parental DNA having a mutation target site, wherein the combinatorial mutagenic primer comprises a variable mutagenic region flanked by a 3′ region that is complementary to a portion of the first parental DNA and a 5′ invariant tail region that is complementary to a portion of the first parental DNA and wherein the first blocking oligonucleotide halts primer extension prior to the 5′ invariant tail region;
(b) adding in a separate, second reaction, a complementary primer and a second blocking oligonucleotide to a second parental DNA that is identical to the first parental DNA, wherein the complementary primer is complementary to the 5′ invariant tail region of the combinatorial mutagenic primer and wherein the second blocking oligonucleotide halts primer extension prior to the mutation target site of the second parental DNA;
(c) synthesizing in the first reaction, by means of at least one cycle of a single-primer linear amplification reaction, a first DNA strand comprising the combinatorial mutagenesis primer having a variable mutagenic region;
(d) synthesizing in the second reaction, by means of at least one cycle of a single-primer linear amplification reaction, a second DNA strand comprising the complementary primer but not comprising a region that is complementary to the variable mutagenic region of the first DNA strand;
(e) combining the reaction products from (c) with the reaction products from (d);
(f) annealing the first DNA strand to the second DNA strand to form a partially double-stranded DNA intermediate; and
(g) extending by means of at least one cycle of a primer extension reaction, the second DNA strand of the partially double-stranded DNA intermediate to copy the variable mutagenic region of the first DNA strand in the presence of third and fourth blocking oligonucleotides, thereby forming a mutagenized combinatorial DNA duplex comprising a variable mutagenic region and further comprising overhanging sticky ends.

2. The method of claim 1 further comprising transforming a host cell with the combinatorial DNA duplex of step (g).

3. The method of claim 1 wherein the single primer linear amplification reaction of steps (c) and (d) are each repeated for at least 20 cycles.

4. The method of claim 1 wherein the primer extension reaction of step (g) is carried out for one cycle.

5. The method of claim 1 wherein the linear amplification reactions of steps (c) and (d) are catalyzed by pfu DNA polymerase.

6. The method of claim 1 wherein the primer extension reaction of step (g) is catalyzed by pfu DNA polymerase.

7. A kit for use in the method of claim 1 comprising a DNA polymerase, and instructions for carrying out the method.

8. The kit of claim 7 further comprising competent or ultracompetent cells.

9. The kit of claim 7 further comprising a DNA vector.

10. The kit of claim 7 further comprising individual nucleotide triphosphates or mixtures of nucleoside triphosphates.

11. A method of using a kit comprising a DNA polymerase and instructions for carrying out the method, in a method comprising the steps of:
(a) adding in a first reaction, a combinatorial mutagenic primer and a first blocking oligonucleotide to a first parental DNA having a mutation target site, wherein the combinatorial mutagenic primer comprises a variable mutagenic region flanked by a 3′ region that is complementary to a portion of the first parental DNA and a 5′ invariant tail region that is complementary to a portion of the first parental DNA and wherein the first blocking oligonucleotide halts primer extension prior to the 5′ invariant tail region;
(b) adding in a separate, second reaction, a complementary primer and a second blocking oligonucleotide to a second parental DNA that is identical to the first parental DNA, wherein the complementary primer is complementary to the 5′ invariant tail region of the combinatorial mutagenic primer and wherein the second blocking oligonucleotide halts primer extension prior to the mutation target site of the second parental DNA;
(c) synthesizing in the first reaction, by means of at least one cycle of a single-primer linear amplification reaction, a first DNA strand comprising the combinatorial mutagenesis primer having a variable mutagenic region;
(d) synthesizing in the second reaction, by means of at least one cycle of a single-primer linear amplification reaction, a second DNA strand comprising the complementary primer but not comprising a region that is complementary to the variable mutagenic region of the first DNA strand;
(e) combining the reaction products from (c) with the reaction products from (d);
(f) annealing the first DNA strand to the second DNA strand to form a partially double-stranded DNA intermediate; and
(g) extending by means of at least one cycle of a primer extension reaction, the second DNA strand of the partially double-stranded DNA intermediate to copy the variable mutagenic region of the first DNA strand in the presence of third and fourth blocking oligonucleotides, thereby forming a mutagenized combinatorial DNA duplex comprising a variable mutagenic region and further comprising overhanging sticky ends.

12. The method of claim 11 wherein the kit further comprises ultracompetent cells.

13. The method of claim 11 further comprising the step of transforming a host cell with the combinatorial DNA duplex of step (g).

14. A method of introducing combinatorial mutations in a DNA sequence of interest, the method comprising the steps of:
(a) adding in a first reaction, a combinatorial mutagenic primer to a parental DNA having a mutation target site, wherein the combinatorial mutagenic primer comprises a variable mutagenic region that corresponds to the mutation target site and wherein the variable mutagenic region is flanked by a 3′ region that is complementary to a portion of the first parental DNA and a 5′ invariant tail region that is complementary to a portion of the first parental DNA;
(b) synthesizing in the first reaction, by means of at least one cycle of a single-primer linear amplification reaction, a DNA strand that is fully complementary to the parental DNA and comprises the variable mutagenic region;
(c) reacting a ligase with the DNA strand to repair nicks and to recircularize the DNA strand after each cycle of the single-primer linear amplification reaction carried out in step (b);
(d) adding a reverse generic primer to the DNA strand of step (c);
(e) synthesizing by means of at least one cycle of primer extension, the reverse complementary strand of the DNA strand;
(f) ligating the reverse complementary strand thereby forming a mutagenized combinatorial DNA duplex comprising a region of variable mutation.

15. The method of claim 14 further comprising the step of transforming a host cell with the combinatorial DNA duplex of step (f).

16. A method for producing combinatorial arrays of viral proteins for use in multiple vaccine production comprising the steps of:
(a) adding in a first reaction, a combinatorial mutagenic primer to a parental DNA having a mutation target site, wherein the combinatorial mutagenic primer comprises a variable mutagenic region that corresponds to the mutation target site of a viral protein and wherein the variable mutagenic region is flanked by a 3′ region that is complementary to a portion of the first parental DNA and a 5′ invariant tail region that is complementary to a portion of the first parental DNA;
(b) synthesizing in the first reaction, by means of at least one cycle of a single-primer linear amplification reaction, a DNA strand that is fully complementary to the parental DNA and comprises the variable mutagenic region;
(c) reacting a ligase with the DNA strand to repair nicks and to recircularize the DNA strand after each cycle of the single-primer linear amplification reaction carried out in step (b);
(d) adding a reverse generic primer to the DNA strand of step (c);
(e) synthesizing by means of at least one cycle of primer extension, the reverse complementary strand of the DNA strand;
(f) ligating the reverse complementary strand thereby forming a combinatorial DNA duplex comprising a variable mutagenic region;
(g) transforming a host cell with the mutagenized combinatorial DNA duplex comprising a region of variable mutation; and
(h) expressing an array of proteins encoded by the combinatorial DNA duplex.

17. A method for producing combinatorial arrays of viral proteins for use vaccine production comprising the steps of:
(a) adding in a first reaction, a combinatorial mutagenic primer and a first blocking oligonucleotide to a first parental DNA having a mutation target site that corresponds to a viral protein, wherein the combinatorial mutagenic primer comprises a variable mutagenic region flanked by a 3′ region that is complementary to a portion of the first parental DNA and a 5′ invariant tail region that is complementary to a portion of the first parental DNA and wherein the first blocking oligonucleotide halts primer extension prior to the 5′ invariant tail region;
(b) adding in a separate, second reaction, a complementary primer and a second blocking oligonucleotide to a second parental DNA that is identical to the first parental DNA, wherein the complementary primer is complementary to the 5′ invariant tail region of the combinatorial mutagenic primer and wherein the second blocking oligonucleotide halts primer extension prior to the mutation target site of the second parental DNA;
(c) synthesizing in the first reaction, by means of at least one cycle of a single-primer linear amplification reaction, a first DNA strand comprising the combinatorial mutagenesis primer having a variable mutagenic region;
(d) synthesizing in the second reaction, by means of at least one cycle of a single-primer linear amplification reaction, a second DNA strand comprising the complementary primer but not comprising a region that is complementary to the variable mutagenic region of the first DNA strand;
(e) combining the reaction products from (c) with the reaction products from (d);
(f) annealing the first DNA strand to the second DNA strand to form a partially double-stranded DNA intermediate;
(g) extending by means of at least one cycle of a primer extension reaction, the second DNA strand of the partially double-stranded DNA intermediate to copy the variable mutagenic region of the first DNA strand in the presence of third and fourth blocking oligonucleotides, thereby forming a mutagenized combinatorial DNA duplex comprising a variable mutagenic region and further comprising overhanging sticky ends;
h) transforming a host cell with the mutagenized combinatorial DNA duplex comprising a variable mutagenic region; and
(i) expressing an array of viral proteins encoded by the mutagenized combinatorial DNA duplex.
